(19) United States
(12) Patent Application Publication
(10) Pub. No.: US 2003/0187500 A1
(43) Pub. Date: Oct. 2, 2003

(54) Conduit cardiac-valve prosthesis and a method for the production thereof

(76) Inventors: Josef Jansen, (US); Rudolf F J Meess, Aachen (DE); Sebastien Willeke, Aachen (DE); Oliver Haller, Aachen (DE); Christoph Classen, Roetgen (DE)

Correspondence Address: THE FIRM OF KARL F ROSS, 5676 RIVERDALE AVENUE, PO BOX 900, RIVERDALE (BRONX), NY, 10471-0900, US

(21) Appl. No.: 10/363,477

(22) PCT Filed: Oct. 2, 2001
(86) PCT No.: PCT/DE01/03809

(30) Foreign Application Priority Data
Oct. 9, 2000 (DE) 100 50 099.4

(51) Int. Cl. B29C 41/14; B29C 41/18; B29C 41/20; B29C 45/14; A61F 2/06; A61F 2/24

(52) U.S. Cl. 623/1.26; 264/305; 264/308; 264/250; 264/259

(57) ABSTRACT

The invention relates to a conduit cardiac-valve prosthesis. Said prosthesis consists of a cylindrical tube or a tube provided with bulbous projections with an integrated support housing comprising a base ring, which bears at least two stanchions that are orientated substantially in the direction of the ring axis and are connected by an arc-shaped wall that fixes flexible leaflets. The invention aims to improve the physiological properties of said prosthesis. To achieve this, the tube, the support housing and the leaflets consist of a single material, preferably polyurethane or another polymer and form a one-piece body.

The invention relates to a tubular cardiac-valve prosthesis comprised of a cylindrical or bulb-shaped tube with an integral support consisting of a base ring that carries at least two posts extending parallel to the ring axis and connected by arcuate walls carrying flexible cusps.

The invention further relates to a method of making a tubular cardiac-valve prosthesis wherein the cusps are made by dipping a male mold having the shape of the cusps several times in a polyurethane solution and, between immersions, drying the polyurethane film on the mold's surfaces, and finally joining the cusps to a tube.

Tubular cardiac-valve prostheses are special cardiac valve prostheses where the cusps are integrated directly into an anatomic, if necessary bulb-shaped, blood-vessel stump.

In order to get a near physiological blood flow in which the flow-dynamic load for the blood corpuscles is acceptable, in the past efforts have been made using plastics that are biocompatible and that because of their mechanical properties allow a largely functional mimicking of a natural cardiac valve. A method of producing an artificial cardiac valve is described in EP 0,114,025. Here valve cusps formed by one or more dippings of an appropriately formed male mold in a polyurethane solution are glued to the valve support. The gluing inherently produces at the joint between the valve cusp and the valve support adhesive adhesions and irregularities that can lead to deposition of cellular blood particles and calcification.

As an alternative to such a procedure, this reference describes how the valve cusps are first formed by immersion of a two-part male mold body and then, after insertion into a female mold part, a sort of valve support is formed also by dipping so that in this step the joints between the valve cusps and the valve support are formed. This method is however relatively expensive because it requires the use of very accurately made molds or thickness variations are created that lead to irregular loading.

In order to avoid these disadvantages EP 0,114,025 proposes dipping a male mold (of stainless steel or plastic)) having surfaces corresponding to the cusps to be formed in a first polymer solution with a viscosity in the neighborhood of 24-192 PA·s at such a slow speed as to prevent bubbles or the like from being created and creating irregularities on the polymer forming on the male mold. After complete immersion the male mold covered with a film is lifted out of the solution and dried.

This process can be repeated until the desired thickness is achieved. Then a preformed valve support is supported in a second polymer solution of lower viscosity in the neighborhood of 1.5-2 Pa·s such that the solution can flow out of lower outlets from inside the valve support. The male mold coated with the cusps is dipped in this second polymer solution and fitted to the valve ring submerged in it. After a short residence time in the solution the male mold with the valve support is raised out of the solution and dried. Thereafter the complete cardiac valve is stripped off the male mold. The thus produced cardiac valve thus is comprised of a support on which several cusps are secured. Such a heart valve, which can also be provided with a suture ring, is intended for installation in a human. Basically and as for example described in WO 97/49,356 such structures are also usable in tubular cardiac-valve implants although this the references do not say how this should be done for tubular cardiac-valve prostheses.

It is thus an object of the present invention to provide a tubular cardiac-valve prosthesis of the above-described type which is improved with respect to its physiological features. In particular such tubular cardiac-valve prostheses should be usable for children.

The above object is achieved in that the tube, the support, and the cusps are made unitarily of a single material, preferably polyurethane or another polymer, and form a one-piece body. The term “a single material” also includes embodiments where for example different polyurethanes of the same material group, if necessary with different mechanical properties, are used. According to the different requirements with respect to flexibility and elasticity of the individual tubular parts, different material thicknesses, different hardnesses, or different strength polyurethanes can be employed. Joints between individual premanufactured parts or the local use of different materials in stressed zones can be avoided by the effective overall use of polyurethane.

Further features of the tubular cardiac-valve prosthesis are described in the dependent claims.

Thus tube ends connected to the support are made of a microporous and elastic polyurethane having a greater elasticity than the support.

If necessary a reinforcement ring preferably made of titanium or a titanium alloy is imbedded in the base ring.

To make the described tubular valve prosthesis, after making the cusps the male mold is fitted to a female mold whose cavity is shaped like the support and the support is cast onto the cusps by injection molding, and thereafter tube ends are either sprayed on both ends of the support or tube ends premade in another mold are adhered to the support, all materials being polyurethane. The tubular cardiac-valve making process thus is constituted by three separate steps, namely the production of the cusps according to the prior art by an alternate dip/tumbling process, followed by two separate injection-molding steps in which at first the support and then the tube ends are molded onto the already formed parts or, if the tube ends are premade, the tube ends are glued on the already made parts (stent with cusps).

Alternatively the tubular valve prosthesis is made according to the invention in that to start with cusp-shaped surfaces of a male mold are coated by individual drops or a stream of a polymer solution or drops or a stream of a viscose polymerizing multicomponent system applied in points, in lines, in strips, in beads, or as a layer to the base body or a support tool, the layer is dried, and the application of drops or of the stream and the subsequent drying is repeated until the desired three-dimensionally shaped polymer body forms the cusp foils. Then the free cusp edges are separated, then a cusp-shaped surface of a female mold is fitted over which forms the downstream part and if necessary also has bulb-shaped bulges. A support is then formed on the male mold by dipping in a polymer solution or application of drops of a continuous stream, a metal ring preferably of titanium or a titanium alloy is slipped over the lower part of this support and it is subsequently imbedded with a polymer by dipping in the appropriate solution alternating with drying, and finally both molds are sprayed to form the tube ends or the tube ends are made separately and affixed by an adhesive, whereby the actual tube with a fine-fiber microporous structure is formed. This fine-fiber microporous structure has as seen flat pores of a size from 20 μm to 80 μm. If necessary oriented fibers can be imbedded in layers, the fiber thickness according to a feature of the invention being between 0.5 μm to 20 μm, preferably 2 μm to 10 μm. According to an alternative embodiment of the invention a fleece can be imbedded from outside in the support so that the entire cross section is formed by a polyurethane film. The outer surface of the support (stent) to which the cusps are secured can be softened before the spraying process with a polymer solution or pure solvent in order to make the support and the fibers better adhere to each other. Since the actual tube as a result of its structure is very elastic, to start with the female mold (with the bulges) can be stripped off and then the male mold pulled out.

An embodiment of the invention is shown in the drawings. Therein:

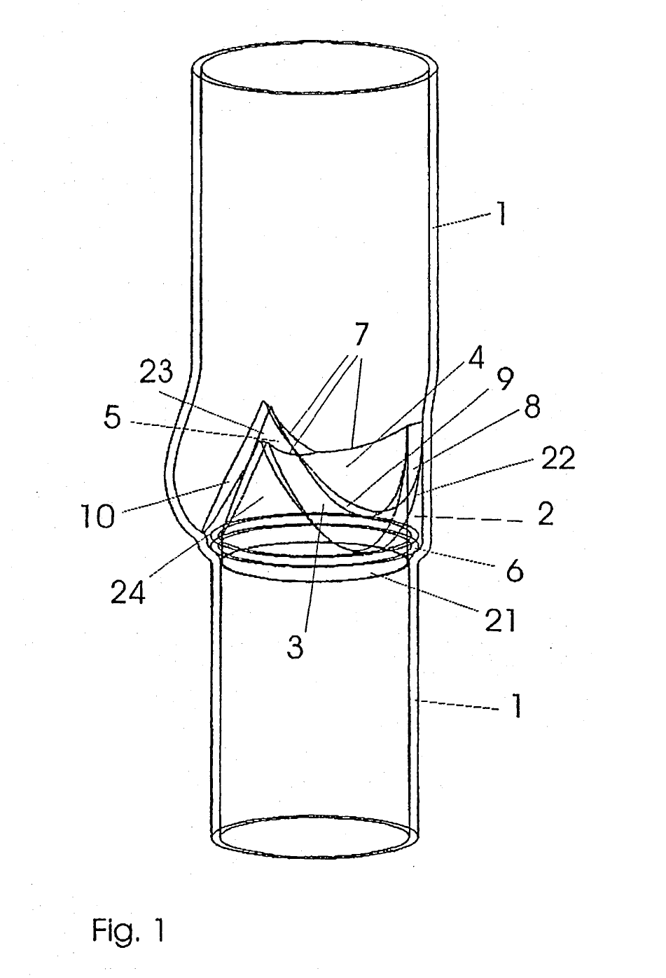

FIG. 1 is a schematic representation of a tubular cardiac-valve prosthesis;

FIG. 2 is a mold for making the tubular cardiac valve;

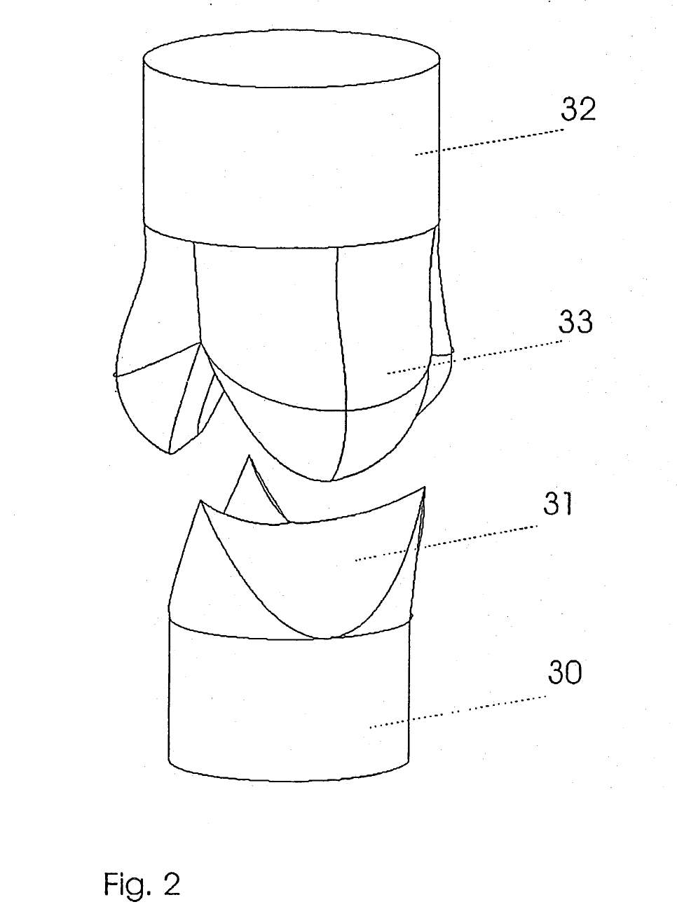

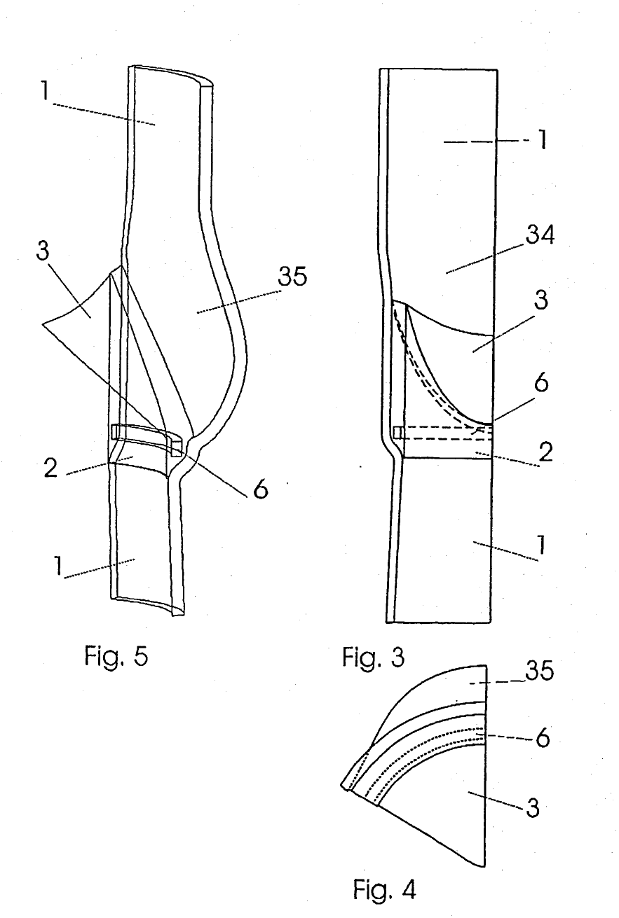

FIG. 3 is a partial longitudinal section through a tubular cardiac-valve prosthesis that is made with the tools of FIG. 2;

FIG. 4 is a segment of a cross section (transverse to the flow direction); and

FIG. 5 is a partial longitudinal section through the tubular cardiac-valve prosthesis according to FIG. 3.

The tubular cardiac-valve prosthesis according to FIG. 1 is comprised of a cylindrical tube 1 with an integral support 2 carrying a base ring 21 as well as three axially extending posts 22, 23, and 24 connected by arcuate walls on which are mounted flexible cusps 3, 4, and 5. All of these parts are made of polyurethane. If necessary a stabilizing titanium ring 6 can be imbedded in the support ring 21.

The support as well as the cusps are made generally as for example described in WO 97/42,356. In particular the aortic valve in this case is part of an integrated tubular cardiac-valve prosthesis that is made as follows:

To start with a male mold as for example of the shape described in EP 0,114,025 is used to make the three cusps 3, 4, and 5. This can be done by repeatedly dipping and drying until the desired cusp thickness is attained. Subsequently the cusps are cut apart along the lines indicated at 7. The male mold is then fitted to a female mold whose cavity has the shape of the support and if necessary the titanium ring 6 has already been installed in holders in the cavity. After injection-molding of the support, which bonds the cusps at their edges 8 and 9 with the support, the assembly formed by the support and the cusps is removed from the mold and put into another female mold in which the end tube parts 1 are also injection molded or, in a separate operation, secured in place by an adhesive. All of the operations use polyurethane, the hardness and strength of the actual materials being varied. The tube ends 1 are formed of microporous elastic polyurethane with a substantially greater elasticity than the polyurethane of the support 2 which in turn is less flexible than the thin-walled cusps 3, 4, and 5.

FIG. 2 shows a male mold 30 that has on its front end mold surfaces 31 that have the desired shape of the three cusps to be made for the aortic cardiac valve. FIG. 2 further shows a female mold 32 that on its front side is complementary to the surfaces 31 and which has lateral bumps 33 that correspond to the bulb shape of later-produced tubular cardiac valve. The body 32 can if necessary have a surface on its front side with which the cusps are engaged along lines toward the stent.

In order to make the tubular cardiac valve first the cusps 3, 4, and 5 are produced on the mold surfaces 31 by dipping or drop-wise application or by application of a stream of a polymer solution, several dippings or doses being necessary. Subsequently the formed cusps are separated along the free cusp edges and the complementary female mold part 32 is fitted over the cusps. Then the cusp joints are thickened at the stent 2 shown mainly in FIG. 7 by casting, one or more dippings, or drop-wise application or stream application of a polymer solution. Meanwhile a titanium ring 6 is slipped over the body 30 and is imbedded by further dipping, molding, or otherwise applied layers.

Finally the bodies 32 and 30 are sprayed so that the actual tube 34 of FIG. 3 is given a fine-fiber microporous structure. The outer surface of the stent 2 can be softened before or after the spraying by means of a polymer solution or pure solvent in order to promote better bonding between the homogenous stent and the tube ends 1. Since the actual tube is very elastic as a result of its structure, first the female mold part 32 with the bulges 33 is removed and then the male mold 30. The bulges are shown at 35.

1. A tubular valve prosthesis comprised of a cylindrical or bulb-shaped tube (1) with an integral support (2) consisting of a base ring (21) that carries at least two posts (22, 23, 24) extending parallel to the ring axis (3) and connected by arcuate walls carrying flexible cusps, characterized in that the tube (1), the support (2), and the cusps (3, 4, and 5) are made unitarily of a single material, preferably polyurethane or another polymer, and form a one-piece body.

2. The tubular valve prosthesis according to claim 1, characterized in that tube ends (1) connected to the support (2) are made of a microporous and elastic polyurethane having a greater elasticity than the support (2), the microporous material having pores that seen flat have a size from 20 μm to 80 μm.

3. The tubular valve prosthesis according to one of claims 1 or 2, characterized in that a reinforcement ring (6) preferably made of titanium or a titanium alloy is imbedded in the base ring (21).

4. A method of making a tubular valve prosthesis according to one of claim 1 to 3, wherein to make the cusps (3, 4, and 5) a male mold with surfaces corresponding to the shape of the cusps is repeatedly dipped in a polyurethane solution and the polyurethane film is dried between immersions and then the cusps (3, 4, and 5) are bonded with a tube (1), characterized in that after making the cusps (3, 4, and 5) the male mold is fitted to a female mold whose cavity is shaped like the support (2) and the support (2) is cast onto the cusps by injection molding, and thereafter tube ends (1) are either sprayed on both ends of the support or tube ends premade in another mold are adhered to the support, all materials being polyurethane

5. A method of making a tubular valve prosthesis according to one of claims 1 to 3, characterized in that to start with cusp-shaped surfaces (31) of a male mold (30) are coated by individual drops or a stream of a polymer solution or drops or a stream of a viscose polymerizing multicomponent system applied in points, in lines, in strips, in beads, or as a layer to the base body or a support tool, the layer is dried, and the application of drops or of the stream and the subsequent drying is repeated until the desired three-dimensionally shaped polymer body forms the cusp foils, that then the free cusp edges are separated, then a cusp-shaped surface of a female mold (32) is fitted over which forms the downstream part and if necessary also has bulb-shaped bulges (33), that a support (2) is then formed on the male mold (30) by dipping in a polymer solution or application of drops of a continuous stream, a metal ring (6) preferably of titanium or a titanium alloy is slipped over the lower part of this support (2) and it is subsequently imbedded with a polymer by dipping in the appropriate solution alternating with drying, and finally both molds (32 and 30) are sprayed to form the tube ends (1) or the tube ends are made separately and affixed by an adhesive.
